(19) United States
(12) Patent Application Publication
(10) Pub. No.: US 2025/0253051 A1
(43) Pub. Date: Aug. 7, 2025

(54) SYSTEMS AND METHODS FOR MULTIMODAL (AUDIO/TEXT/VIDEO) AND AUTOMATED CHAT-BOTS FOR SCREENING AND MONITORING OF MENTAL HEALTH CONDITIONS

(71) Applicant: AIBERRY, INC., Bellevue, WA (US)

(72) Inventors: Ezra Morse, Qualicum Beach (CA); Linda S. Chung, Bellevue, WA (US); Jason Dee Shumake, Austin, TX (US); Lior Auslander, Bellevue, WA (US); Ovidiu Roman, Tacoma, WA (US); Hassan Ugail, Ilkley (GB); Ali Ahmed M. Elmahmudi, Bradford (GB); Aliyu Abubakar, Bradford (GB); Ahmed Aman Ibrahim, Male (MV); Hong Pengfei, Singapore (SG); Michael Collins Mullarkey, Denver, CO (US); Matthew Dominic Matze, Tampa, FL (US); Mona Vana Schreiber, Kirkland, WA (US)

(73) Assignee: AIBERRY, INC., Bellevue, WA (US)

(21) Appl. No.: 18/960,358

(22) Filed: Nov. 26, 2024

Related U.S. Application Data
(63) Continuation-in-part of application No. PCT/US2023/024211, filed on Jun. 1, 2023.
(63) Continuation-in-part of application No. PCT/US2023/024289, filed on Jun. 2, 2023.
(63) Continuation-in-part of application No. PCT/US2023/024260, filed on Jun. 2, 2023.
(63) Continuation-in-part of application No. 18/959,400, filed on Nov. 25, 2024.
Application No. 18/959,400 is a continuation-in-part of application No. PCT/US2023/024211, filed on Jun. 1, 2023.
Application No. PCT/US2023/024211 is a continuation-in-part of application No. PCT/US2023/024289, filed on Jun. 2, 2023.
Application No. PCT/US2023/024289 is a continuation-in-part of application No. PCT/US2023/024260, filed on Jun. 2, 2023.
(60) Provisional application No. 63/604,785, filed on Nov. 30, 2023.
(60) Provisional application No. 63/604,803, filed on Nov. 30, 2023.
(60) Provisional application No. 63/604,823, filed on Nov. 30, 2023.
(60) Provisional application No. 63/604,828, filed on Nov. 30, 2023.
(60) Provisional application No. 63/604,834, filed on Nov. 30, 2023.
(60) Provisional application No. 63/604,840, filed on Nov. 30, 2023.
(60) Provisional application No. 63/604,785, filed on Nov. 30, 2023.
(60) Provisional application No. 63/604,803, filed on Nov. 30, 2023.
(60) Provisional application No. 63/604,823, filed on Nov. 30, 2023.
(60) Provisional application No. 63/604,828, filed on Nov. 30, 2023.
(60) Provisional application No. 63/604,834, filed on Nov. 30, 2023.
(60) Provisional application No. 63/604,840, filed on Nov. 30, 2023.

(51) Int. Cl. G16H 50/30; G16H 10/20; G16H 30/40; G16H 40/67; G16H 50/20

(52) U.S. Cl. CPC G16H 50/30; G16H 10/20; G16H 30/40; G16H 40/67; G16H 50/20

(57) ABSTRACT

A computer-implemented method includes receiving from a user over a network a media file including a recorded patient screening interview, receiving from the user over the network data comprising one or more responses provided by the patient to a mental health questionnaire, generating a transcription of audio associated with the media file, presenting to the patient via a graphical user interface (GUI) a first question of the set of questions, performing video sentiment analysis to generate a second data set, presenting to the patient via a graphical user interface (GUI) the second set of questions based on at least one of the transcription, first and/or second data, generating an artificial intelligence model configured to determine a score characterizing a relevance of a mental health topic to the patient and provide predicted risk levels for one or more mental health conditions based on the patient's first and second set of answers.

### PRIORITY CLAIM AND CROSS-REFERENCE TO RELATED APPLICATIONS

This application is a Continuation-in-Part under 35 U.S.C. §§ 120 and 365 (c) of International Application No. PCT/US2023/024211 filed Jun. 1, 2023, International Application Serial No. PCT/US2023/024289 filed Jun. 2, 2023 and International Application Serial No. PCT/US2023/024260, filed Jun. 2, 2023, which claim priority to U.S. Provisional Patent Application Nos. 63/348,946, 63/348,955, 63/348,964, 63/348,973, 63/348,991, 63/348,996, and 63/349,007, all filed Jun. 3, 2022.

This application is also a Continuation-in-Part of and claims priority to U.S. patent application Ser. No. 18/959,400 filed Nov. 25, 2024, which application claims priority to U.S. Provisional Patent Application Ser. No. 63/604,785 filed Nov. 30, 2023, U.S. Provisional Patent Application Ser. No. 63/604,803 filed Nov. 30, 2023, U.S. Provisional Patent Application Ser. No. 63/604,823 filed Nov. 30, 2023, U.S. Provisional Patent Application Ser. No. 63/604,828 filed Nov. 30, 2023, U.S. Provisional Patent Application Ser. No. 63/604,834 filed Nov. 30, 2023, and U.S. Provisional Patent Application Ser. No. 63/604,840 filed Nov. 30, 2023. the entireties of all of which are hereby incorporated by reference as if fully set forth herein.

### COPYRIGHT NOTICE

This disclosure is protected under United States and/or International Copyright Laws.© 2024 AIBERRY. INC., All Rights Reserved. A portion of the disclosure of this patent document contains material which is subject to copyright protection. The copyright owner has no objection to the facsimile reproduction by anyone of the patent document or the patent disclosure, as it appears in the Patent and/or Trademark Office patent file or records, but otherwise reserves all copyrights whatsoever.

### BACKGROUND

One of the biggest challenges in this area is that it has historically not been a precise science. Today, most of the practitioners use self-reported scores from patients using standard mental health questionnaires, such as PHQ-9, QIDS, HRSD, BDI, CES-D, etc. For example, there are many problems with depression measurement. A first major problem is heterogenous content. A review of 7 commonly used scales for depression found they contain 52 disparate symptoms, 40% of which appear in only 1 of the scales. This is not surprising given that these instruments were developed by scholars working in distinct settings toward distinct goals, and in the absence of a unifying theory of depression. Not surprisingly, correlations between different scales are often only around 0.5.

A second major problem is that irrelevant response processes influence depression measurement. For example, self-reported symptoms of depression tend to be more severe than observer ratings. One reason for this is that clinicians may not score symptoms endorsed in self-report scales if they can be attributed to external causes. For example, getting little sleep when caring for a newborn may lead someone to endorse a high score on items related to sleep problems, which in this case should not factor into a calculation of depression severity. Alternatively, some individuals may be more candid on a self-report questionnaire than they are in a clinical interview.

A third major problem is the common practice of summing all items from these scales and using a threshold to determine the presence of MDD, despite considerable evidence that depression is not categorical but rather exists on a continuum from healthy to severely depressed. Moreover, these sum scores weight all symptoms equally as if they were all interchangeable indicators of a single underlying cause, namely, depression. This assumption is demonstrably false given a broad set of empirical findings showing that depression scales are measuring multiple dimensions, not just one, and that the number and nature of the constructs being measured shift across contexts.

Additionally, one of the challenges facing mental health professionals is finding off-the-shelf models for video sentiment analysis. Put simply, video sentiment analysis is the ability to determine certain sentiments of a patient like “Happy”, “Sad”, “Neutral” that is portrayed in the video by analyzing the video and determining those sentiments. Most of the extant prior art models suffer from overfitting and provide inaccurate results which are strongly biased towards one or two sentiments. Overfitting can occur when a machine learning model has become too attuned to the data on which it was premised or trained and therefore loses its applicability to any other dataset.

One of the reasons for this is that most of the existing models are based on staged data, mostly actors posing to the camera. In addition, there is no gold standard that is based on true, valid and accurate mental health related data. Embodiments of the invention described herein address and solve the foregoing problems.

### BRIEF DESCRIPTION OF THE DRAWING FIGURES

FIG. 1 illustrates components of a system that can be used in data collection for model training purposes to execute a process according to one or more embodiments.

FIG. 2 illustrates the utilization of an AI model in an inference process according to one or more embodiments.

FIG. 3 illustrates an inference process flow diagram according to one or more embodiments.

FIG. 4 illustrates an entity relationship diagram that shows the structure of a proprietary database that is used to store data for training purposes according to one or more embodiments.

FIG. 5 is a simple diagram illustrating the concepts of overfitting and underfitting according to one or more embodiments.

FIG. 6 illustrates how various features are ranked according to one or more embodiments.

FIG. 7 illustrates a process of obtaining from atomic predictions a final combined score via a fusion process according to one or more embodiments.

FIG. 8 illustrates a means of plotting patient screening over a period allowing the practitioner to easily view changes in screening scores according to one or more embodiments.

FIG. 9 illustrates solution capability of keeping track of all historical screenings according to one or more embodiments.

FIG. 10 illustrates the ability of one or more embodiments to identify inconsistencies between self-reported scores and AI-model prediction scores according to one or more embodiments.

FIG. 11 illustrates a sloped line that demonstrates good prediction according to one or more embodiments.

FIG. 12 illustrates a sloped line that demonstrates sub-optimal prediction according to one or more embodiments.

FIG. 13 illustrates an analytical screenshot according to one or more embodiments.

FIG. 14 illustrates an example of plotting audio modality results and comparing self-reported score versus AI models according to one or more embodiments.

FIG. 15 illustrates a native transcript according to one or more embodiments.

FIG. 16 illustrates a conversational structure according to one or more embodiments.

FIG. 17 illustrates a digital representation of a standard mental health questionnaire such as PHQ-9 according to one or more embodiments.

FIG. 18 illustrates the results of an AI-based mental health screening according to one or more embodiments.

FIG. 19 illustrates the results of a process of identifying, analyzing, and reacting to score outliers according to one or more embodiments.

FIG. 20 illustrates a chart output from a process according to one or more embodiments.

FIG. 21 illustrates an automated chat-bot graphical user interface (GUI) according to one or more embodiments driving a self-screening.

FIG. 22 illustrates an automated chat-bot GUI according to one or more embodiments driving a telehealth session.

FIG. 23 illustrates a sloped line that demonstrates good prediction according to one or more embodiments.

FIG. 24 illustrates a sloped line that demonstrates sub-optimal prediction according to one or more embodiments.

### DETAILED DESCRIPTION

One or more embodiments include a method by which data is collected and processed for AI model training purposes for which are generated AI models that predict risk levels for certain mental health conditions (e.g., depression). A method according to one or more embodiments collects data through a short video recording:
Text—What do we say?
Audio—How do we say it?
Video—Focusing on facial expressions. In an embodiment, use of video sentiment analysis.

A method according to one or more embodiments uses a multimodal approach that enhances prediction accuracy by utilizing three independent sources of data.

To build efficient and scalable AI models that will be less likely to suffer from overfitting, there are several guidelines that advantageously are well executed, which is why one or more embodiments include a robust and unique infrastructure focusing on:
Quality of input data into the models.
Accuracy of extracted features into the training model.
Clear separation of speakers (speaker diarization).
Cleaning up quiet sections (none speaking) from audio stream.
Defining conversation context.
Features selection and effectiveness measurement.
Ways of analyzing the output and understanding correlations between input data and prediction accuracy to enable an effective repeatable process.
Solid statistical model to allow for effective and realistic folds definitions for the purpose of model training.

### Capability 1—Infrastructure to Support Automated Collection and Processing of Training Data.

This capability describes the infrastructure in support of AI models training according to one or more embodiments.

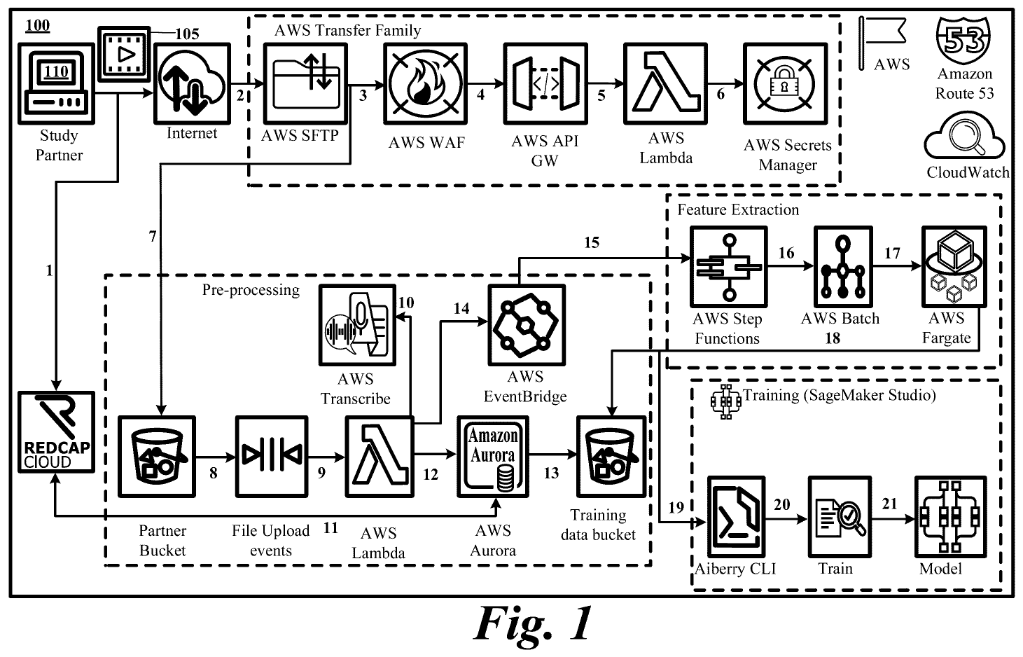

The infrastructure according to one or more embodiments can be built on Amazon Web Services (AWS) foundations. FIG. 1 illustrates components of a system 100 that can be used in data collection for model training purposes to execute a process according to one or more embodiments. One or more embodiments can leverage a REDCap Cloud solution to manage its clinical studies with various study partners and that data can be automatically loaded into the system. As part of a clinical study, one or more embodiments collect various demographic information, video recorded interviews as well as self-reported mental health questionnaires data such as PHQ-9, QIDS, GAD-7, CES-D, etc.

Referring to FIG. 1, at a step 1, a study partner 110 keeps track of participant demographic data and questionnaires in, for example, a REDCap Cloud. REDCap is a third-party product that helps to manage clinical studies in a secure and HIPAA (Health Insurance Portability and Accountability Act of 1996 (HIPAA) (Public Law 104-191, see 42 U.S.C. § 1320d-2) compliant manner.

At a step 2, the study partner 110 uploads one or more videos 105, which may include all multimedia elements including audio, to an sFTP server over a network, such as the Internet. sFTP is a standard AWS service that allows one to transfer files in a secure manner. This is used to transfer data in a secure manner to a back-end portion of one or more embodiments.

At a step 3, an authentication request is processed through a firewall with internet protocol whitelisting rules. AWS WAF helps to protect against common web exploits and bots that can affect availability, compromise security, and/or consume excessive resources.

At a step 4, the authentication process is then delegated to a custom authentication method exposed via API gateway. AWS API Gateway is a fully managed service that makes it easy to create, publish, maintain, monitor, and secure APIs at any scale. This is where APIs are hosted according to one or more embodiments.

At a step 5, the API GW invokes a serverless function to authenticate the user. AWS Lambda is a serverless, event-driven compute service that allows one to run code for virtually any type of application or backend service without provisioning or managing servers. This is what is used to host small functions in a processing pipeline according to one or more embodiments.

At a step 6, the serverless function uses a secure location to authenticate the user and to identify the bucket allocated for the study partner 110. AWS Secrets manager helps to manage, retrieve, and rotate database credentials, API keys, and other secrets throughout their lifecycles. This is where sensitive access information is stored in one or more embodiments.

At a step 7, and if succeeded, the sFTP server uploads the media files to the identified study partner bucket. Study partner buckets are AWS S3 buckets that are used according to one or more embodiments to store data coming from the study partners 110. The data may be transferred using sFTP.

At a step 8, file uploads generate cloud events. File upload events are used to listen to data upload events and then take necessary actions to trigger an automated pipeline to process the files.

At a step 9, a serverless function processes the upload events. AWS Lambda in this context are functions that compose the file processing pipeline and that trigger various actions to happen in a prescribed order.

At a step 10, if the file includes an audio file, then system 100 extracts a corresponding transcript. AWS transcribe is a speech to text service that is used to create a transcription from the screening interview according to one or more embodiments.

At a step 11, the demographic data as well as the questionnaire answers are retrieved from REDCap Cloud. AWS Lambda in this context is used to fetch supplemental data from the REDCap Cloud system according to one or more embodiments.

At a step 12, all processed information is stored in the database. AWS Aurora is a global-scale relational database service built for the cloud with full MySQL and PostgreSQL compatibility. This is used to store all data in a designated database according to one or more embodiments.

At a step 13, all media files including the transcripts are moved to the training bucket. Training data bucket is an S3 bucket used to store all the relevant file after the pipeline processing (outputs) according to one or more embodiments.

At a step 14, an upload completion event is triggered. AWS Eventbridge is a serverless event bus that ingests data from your own apps, SaaS apps, and AWS services and routes that data to targets. This service is used to created notification events between various processes in the pipeline according to one or more embodiments.

At a step 15, the event triggers a step function that orchestrates the process of text, audio and video feature extraction. AWS step functions as a visual workflow service that helps developers use AWS services to build distributed applications, automate processes, orchestrate microservices, and create pipelines. This capability is used to construct a feature extraction process from the screening interview according to one or more embodiments.

At a step 16, a batch job is triggered to extract the features.

At a step 17, the batch job runs on a Fargate compute cluster, leveraging spot instances.

At a step 18, and when completed, the extracted features are uploaded to the bucket. Regarding steps 16-18, these are the specific components that are built into the step function pipeline that are doing the actual feature extraction according to one or more embodiments.

At a step 19, a command line interface is provided to retrieve participant data. A command line interface is a proprietary technology according to one or more embodiments able to pull data in various slices for the purpose of AI training processes.

At a step 20, training sessions are executed using infrastructure managed by, for example, SageMaker. This is a fully managed machine learning service used to train and generate an AI model according to one or more embodiments.

At a step 21, models are published that are used in the inference process.

Once data is uploaded from a study partner, it can be post-processed and relevant TEXT/VIDEO/AUDIO features can be extracted utilizing proprietary methods described below herein. In addition, all the information is stored in a database so that it can be accessed in perpetuity for a model training process according to one or more embodiments.

For model training purposes one or more embodiments can leverage AWS Sagemaker and one or more embodiments include an interface that will allow extraction of relevant training data (based on defined selection criteria) into the Sagemaker studio.

In order to train an AI model, one needs to decide on the features that will be part of the training. The features decision is advantageous for enabling the model accuracy. Selecting the wrong features will result in a poor model and also selecting too few or too many features in order to pass a successful test will likely result in a model under/over fitting which means that the model won't perform well at scale or when dealing with new data. For every modality, AI models according to one or more embodiments have a specific set of features such as, for example, physical audio characteristics such as Jitter, Shimmer, Articulation, etc. The specific list of features can change over time in response to subsequent cycles of model training.

One or more embodiments provide contextual conversation and a mechanism for measuring the effectiveness and continuous improvement for such conversation. As part of our studies, contextual conversation is more likely to produce more accurate predictions of mental health issues than non-contextual conversation.

One or more embodiments not only create a contextual conversation but also:

Provides domain specific prediction models with a proprietary fusion process to combine all scoring results.

Ensures that each domain is managed in a way that the conversation is natural and has a randomization mechanism to assist in better coverage. One or more embodiments provide a “360 degree” view and assessment during the screening process which may be done via dipping into various domains. Since according to one or more embodiments a screening interview is changing between screenings, one is able to collect a well-rounded picture based on multiple, diverse data points.

A mechanism to measure the effectiveness of each domain in the overall prediction process.

As a result, one or more embodiments provide for a broader and more complete prediction score.

For purposes of the discussion herein, the term “domain” may refer in non-limiting fashion to topics of analysis including one or more of Sleep, Appetite, General Wellbeing, Anxiety, Diet, Interests, etc. The term “modality” may refer in non-limiting fashion to video, audio and text. In the context of one or more embodiments of the invention, multiple modalities are used, and each modality provides a prediction for each domain.

One or more embodiments include a method of defining domain/topics and connecting those to screening process and questions that can assist in optimizing the reactions/responses from patients to improve accuracy of data going into AI modeling.

This capability covers the methodology according to one or more embodiments to analyze and improve the effectiveness of the conversation screening to improve prediction accuracy.

The first step of the process is conducting the study interviews. The interviews may be done using teleconference and are recorded. The interviewers may use randomized screening scripts to go through the core screening domains. More particularly, a team of study coordinators can identify qualified participants and conduct a screening interview with them or assign them a self-screening interview done by a chat-bot, which may be referred to herein as “Botberry.” As people are recruited to this study, proper statistical distribution across gender, age, perceived symptoms severity, race, etc. is maintained. The participants can undergo a screening process and can also self-report their condition using a standard form for depression assessment such as, e.g., PHQ-9.

Once study data (e.g., video interviews) is loaded into storage, the data can be processed and relevant features for each of the modalities can be extracted.

The next step is running the various models across all modalities aligned with the automatic folds definition that uses a proprietary method according to one or more embodiments to ensure statistically sound data distribution to avoid AI models overfitting. In this step, all interview videos are run through an inference process to obtain a numerical score.

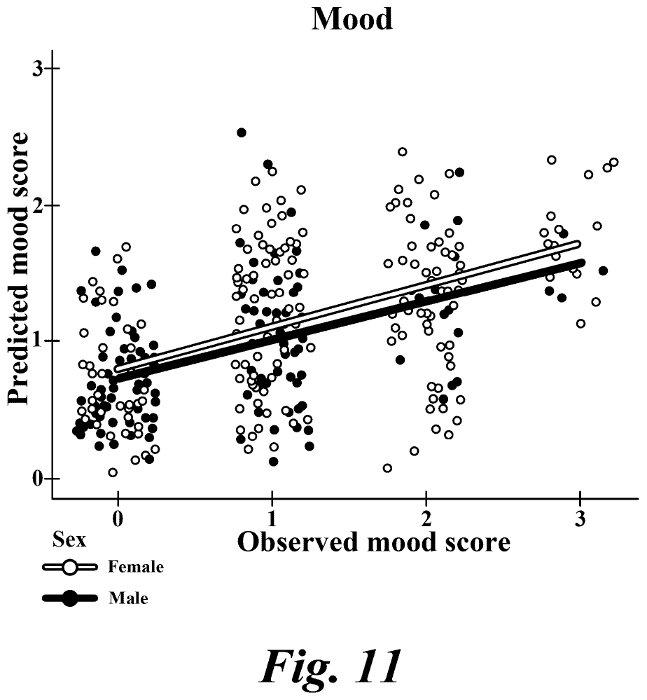

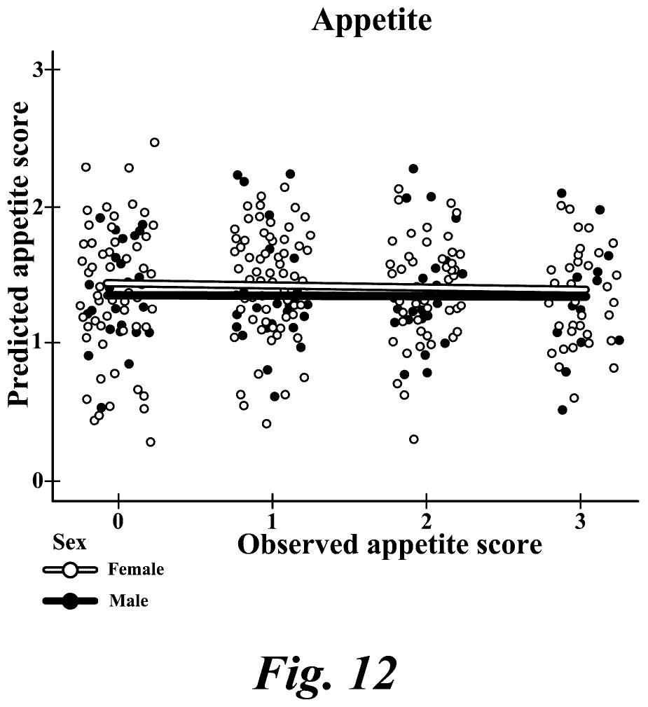

The next step of the process is to plot all the test data and compare self-reported scores provided by the study participants and the score coming from the AI models. In this step, a plot is constructed in which on one axis there is a self-reported (observed) score and on the other one is the score predicted by one or more embodiments. One or more embodiments seek a sloped line that will demonstrate good prediction (e.g., FIG. 11) as opposed to a flat line (e.g., FIG. 12).

The final step is to identify areas for improvement. Based on the data collected in the previous step, one can now design a revision to the areas where one does not see sufficiently good predictions.

Through detailed analysis, one can identify the effectiveness of each model looking at gender-specific behavior, effectiveness of each feature used, etc.

This process assists in designing the fusion process which is where all individual predictions are merged into a combined prediction score.

As illustrated in FIGS. 11 and 12, the difference between a high-relevancy topic (Mood shown in FIG. 11) showing a pronounced slope line (correlation) between reported and AI predicted scores and a low-relevancy topic (Appetite shown in FIG. 12) showing a flat line and as such low correlation between reported and AI predicted scores.

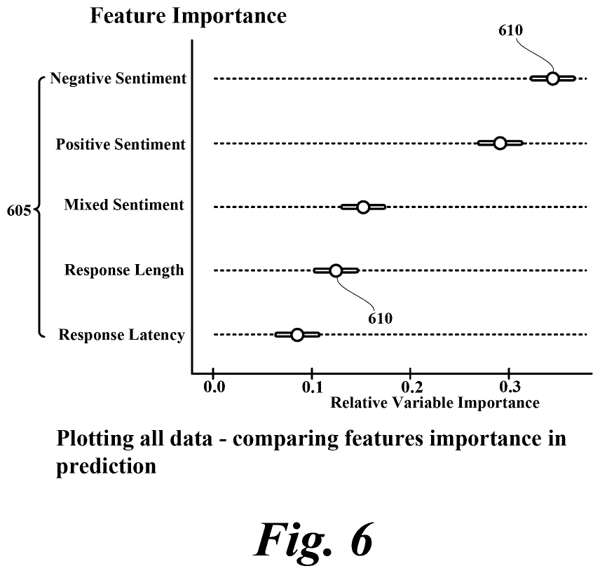

FIG. 6 illustrates a graph 600 including a graphical display of elements 610 that show how various features 605 are ranked. Distinguishing displayed features such as different colors, shading and/or length of each element 610 may be associated with, for example, range of relative variable importance of the displayed features. This helps to further tune the AI model and helps with the decision on which features to use, whether addition or removal of certain features is helping/hurting model accuracy, etc. As part of building an AI model, one needs to decide which features they are going to define, extract and train on. As one trains and then tests a model, one can then measure how effective are the features selected and rank them (which is illustrated in FIG. 6). Once one has this data, one can decide if one wants to keep certain features or drop them. The more features one has that add no value, the more unnecessarily complicated one's process is. As such, this data helps to train the AI algorithm.

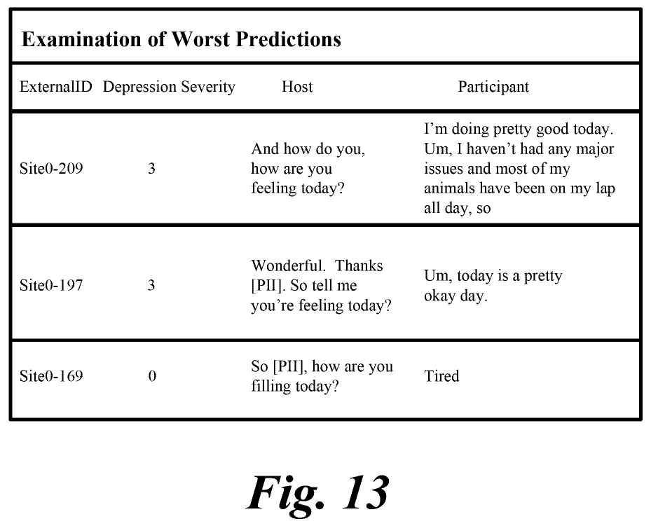

In addition to what is illustrated in FIGS. 11-12 and 6, the most granular level of analysis is illustrated in FIG. 13. This method allows one to quickly identify discrepancies between reported scores and specific responses provided by the study participants. In FIG. 13 one can see study participants in which there is bad correlation between the DepressionSeverity number (Higher is more severe) and the Participant response. As such, and for example, Site0-197 said “Um today is a pretty okay day” which could be assessed anywhere from neutral to positive, but the DepressionSeverity is showing 3 which is very negative. Thus, using such information, analysts can perform a deep analysis and understand where there might be problems with the model and help to correct them.

This helps the team to determine whether corrections are required to the models or alternatively, through a clinical assessment, to determine whether the reported scores are not correctly representing the true state of mind of the study participant. Identifying such discrepancies between self-reported scores and AI model scores is a key capability and benefit of the solution and infrastructure of successive approximations, in which an iterative exchange between depression instruments and AI predictions advances us toward more comprehensive and reliable measurement.

One or more embodiments include the ability to demonstrate and measure correlations between topics/questions and the responses we are getting from patients for different modalities.

To supplement above capabilities, one or more embodiments expand across multiple modalities. Understanding how certain domains work across study participants is advantageous. This also helps one to understand gender specific aspects across the various modalities and domains, specifically when dealing with physical attributes.

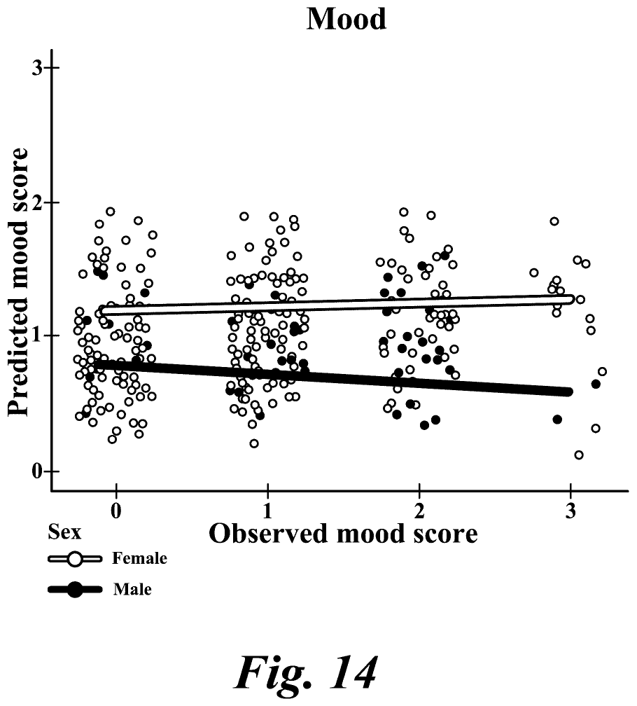

In FIG. 14, shown is an example of plotting AUDIO modality results and comparing self-reported score versus AI models. FIG. 14 provides an example of how one can understand the effectiveness of each atomic domain/modality combination. Also as observed from FIG. 14, it is suggested that this might be a gender-specific model since there is enough difference in the responses of each gender.

In essence the predicted score is plotted against the self-reported score and alignment is sought between the two. A nicely sloped line (45 degrees may be considered the optimal) will demonstrate a high degree of correlation between the two, which means that the model comported to the control data is well-aligned. This can help to assess how each domain is performing and improve the performance of each by means of selecting different features etc.

The infrastructure allows for full tracking and monitoring of the E2E process and applies relevant security measures and procedures.

### Capability 2—Infrastructure to Support Automated Inference Process.

This capability describes an inference process according to one or more embodiments and the infrastructure that supports it.

The inference process relies on the AI models created during execution of Capability 1 described above. This process describes how they are invoked, and the output generates a prediction.

This process according to one or more embodiments may be built on AWS infrastructure and may utilize various AWS services to support a fully automated process:

Receiving the input in the form of a video or audio recording.

Transcribing it, utilizing Aiberry proprietary methods and the processes described herein supported by AWS transcribe service.

To increase the chances of a good AI model, there are few principals that have proven to be key success factors.
1. Diarization—The method by which one or more embodiments identify and separate speakers to understand who said what.
2. VAD (Voice Activity Detection)—Cleaning out any non-speaking segments

To enable these two activities one or more embodiments include a method to transform a native transcript into a proprietary structure of questions and answers. This process is now described below herein.

One or more embodiments provide a solution to transform native transcript into a conversational transcript that is used for driving AI models. The process may be referred to as speaker diarization and is a combination of speaker segmentation and speaker clustering. One or more embodiments provide a proprietary algorithm to accomplish these objectives and reconstruct the input file into a conversational structure with a clear questions-and-answers structure to represent the essence of a dialogue between a patient and practitioner and to better structure a self-screening process.

The purpose of this algorithm is to convert a native transcript into a “true” conversation of one question vs. one combined answer. In the course of executing this algorithm we are dealing with situations in which the speakers speak over each other, dealing with small interruptions such as “Hmmm”, “Yep” and other vague and/or irrelevant expressions that actually breaks the sequence of the conversations and breaks the context of the responses. As such, the algorithm deals with cleanup of irrelevant text and bundling responses into a coherent well-structured response that then can be analyzed by an inference process to deduce sentiment and other key insights. The result is a clear one question vs. one answer structure with calculated time stamps, speaking vs. non-speaking tags and more.

In technical terms, the algorithm takes a native transcript as an input, processes the transcript file and then constructs a clear structure of one host question vs. one participant answer. While doing that, it is noting time stamps, speaking vs. nonspeaking expressions, cleaning up irrelevant text, analyzing the topic of the question etc. that is then written to a new format file that is used in downstream processing.

Having an effective and accurate diarization process is a cornerstone to preparing the data both for training and inference processes. Aside from just speaker separation and clustering, the diarization process according to one or more embodiments also generates a high quantity of additional meta-data to the conversation that is advantageous for effective feature extraction processes, for example removing quiet audio periods.

A capability of one or more embodiments includes proprietary method developed for speaker diarization according to one or more embodiments.

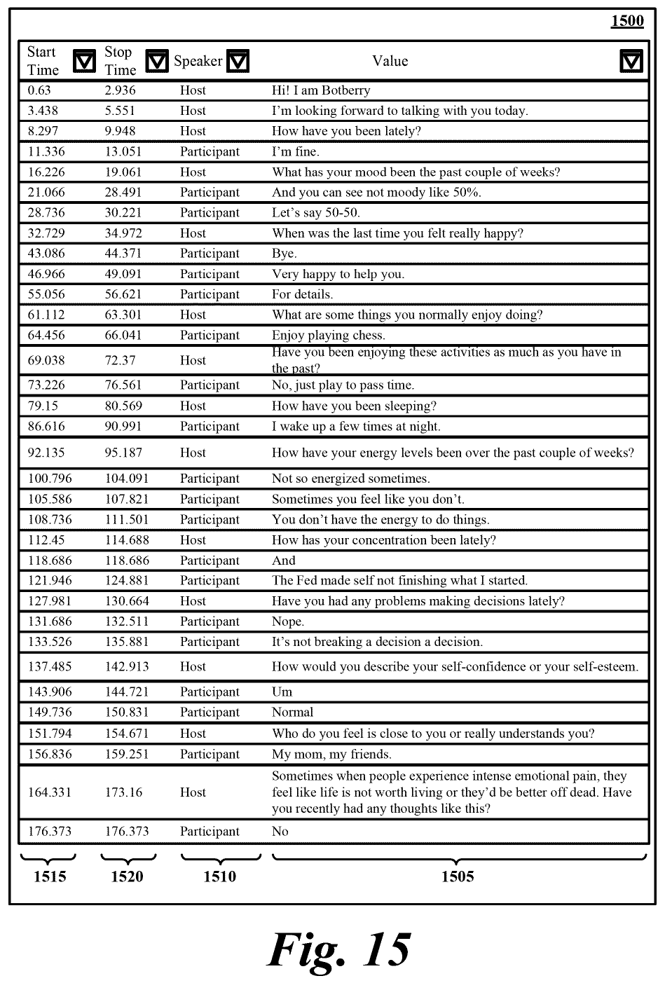

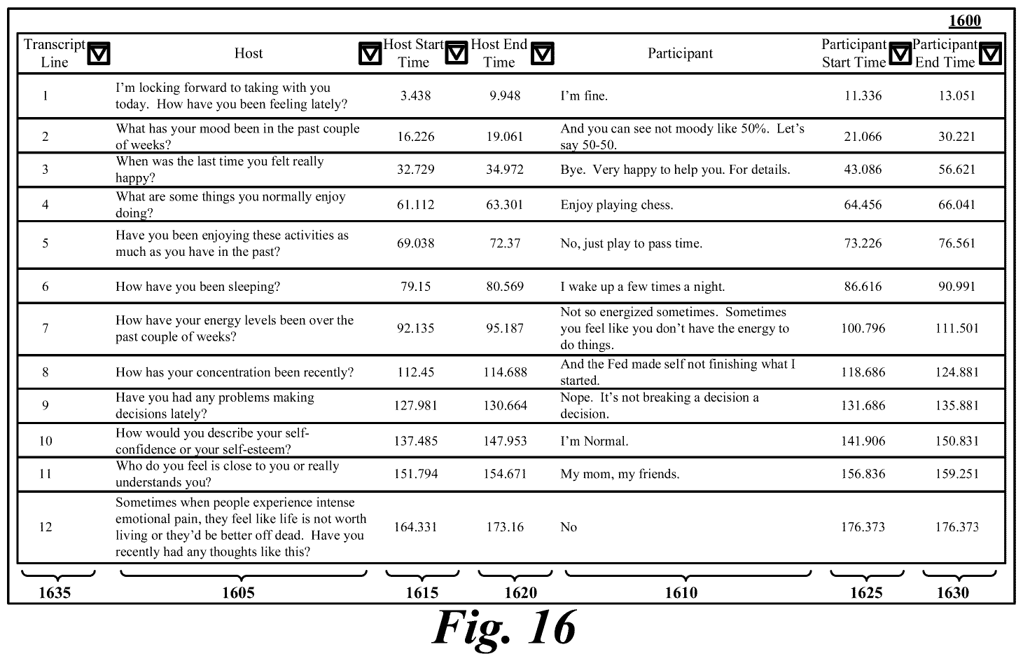

This method uses a native transcript illustrated in FIG. 15 that comes out of the screening session and transposes it into a conversational structure as illustrated in FIG. 16.

FIG. 15 illustrates a native transcript 1500 as alluded to above. Transcript 100 includes a set of value fields 1505 that indicate what was said by a speaker participating in the screening session, a set of speaker fields 1510 indicating the identity of each speaker of a corresponding statement indicated in the value fields, a set of start time fields 1515 including time stamps of when each such statement began and a set of stop time fields 1520 including time stamps of when each such statement ended.

FIG. 16 illustrates a conversational structure 1600 as above alluded to. Structure 1600 includes a set of host fields 1605 that indicate what was said by the host (typically a mental health practitioner) participating in the screening session, a set of participant fields 1610 that indicate what was said by the participant (typically a patient) participating in the screening session, a set of host start time fields 1615 including time stamps of when each host statement began, a set of host stop time fields 1620 including time stamps of when each host statement ended, a set of participant start time fields 1625 including time stamps of when each participant statement began, and a set of participant stop time fields 1630 including time stamps of when each participant statement ended.

This method of classification and clustering is an advantageous component in the proprietary method for features extraction according to one or more embodiments.

As part of this transformation, one or more embodiments also clearly annotates section of speaking VS sections of non-speaking and groups together fragments of responses to a coherent full response that can then be further analyzed and processed as a whole.

Extracting the features for TEXT/AUDIO/VIDEO (as described in Capability 1).

Invoking the various models to get a modality level scoring.

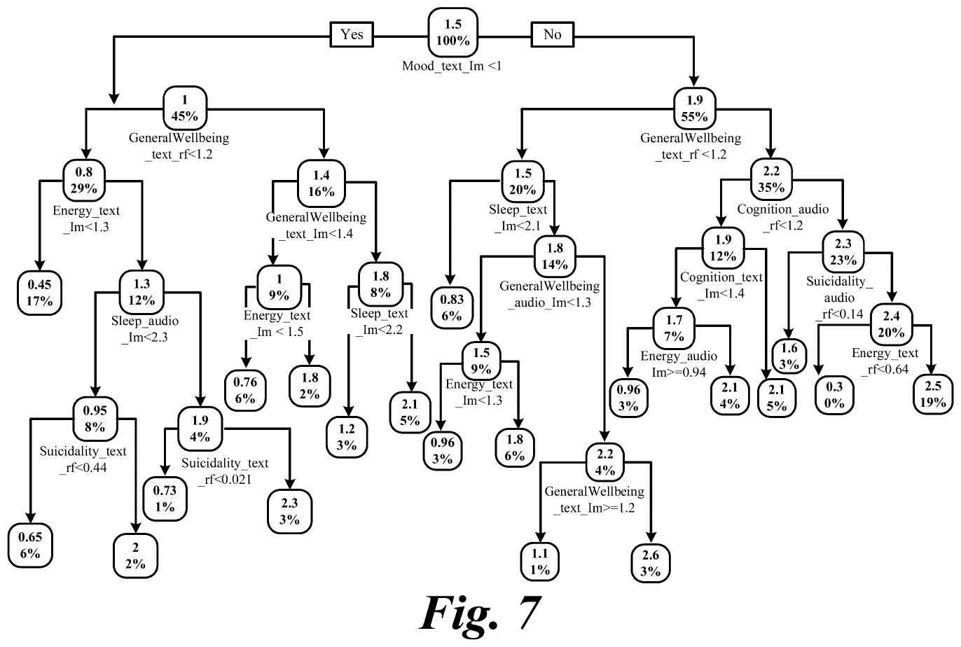

A proprietary fusion process coupled with the processes described herein generates a final prediction score for risk levels for certain mental health conditions. The fusion process according to one or more embodiments is the process in which one takes an inference response from each of the modalities and domain and constructs a final combine score for the screening. This is also further illustrated in FIG. 7.

Based on information collected during the training process regarding the effectiveness of certain features and domain as being an accurate predictors (e.g., FIG. 6), one can then feed all that information into a statistical model that produces a linear function that sets the respective value of each of the parameters in the overall final score formula. This function takes into account the effectiveness of the prediction and correspondingly sets its contribution value.

The final formula takes into account all predictors across modalities and domains such that it is very resilient to situations where one or two predictions might be absent.

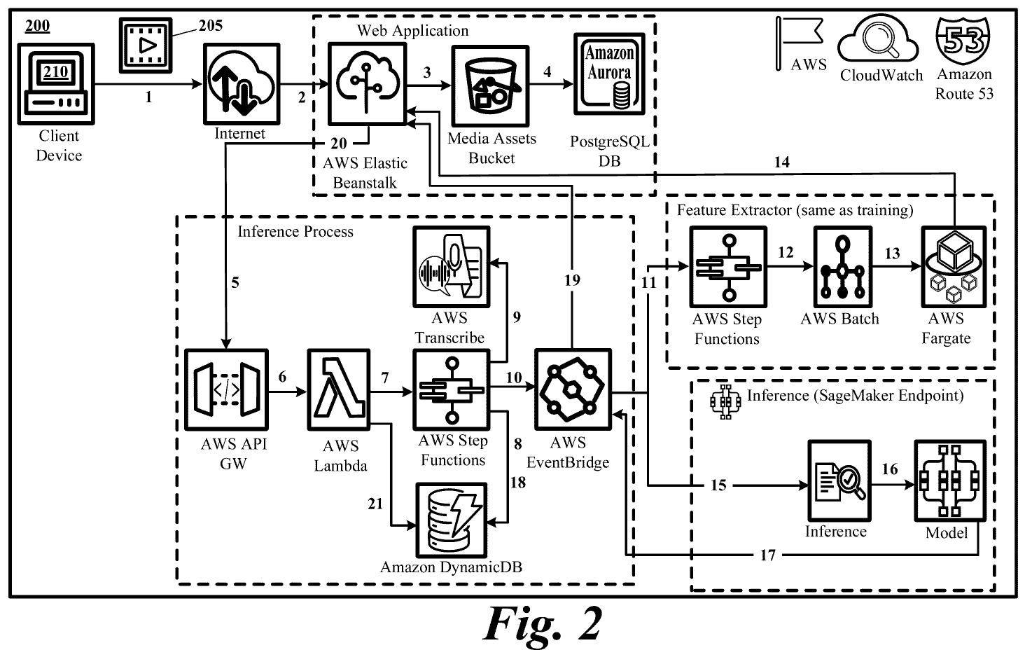

According to one or more embodiments, FIG. 2 may be considered a subset of FIG. 1. In essence, FIG. 1 describes the AI training process and FIG. 2 illustrates the utilization of the AI model in an inference process.

Referring to FIG. 2, at a step 22, a user uses an application, such as a WebApp, according to one or more embodiments to record a media file 205 that may include video and audio assets. Such can be done on a processing device 210 to conduct the screening interview.

At a step 23, the customer requests a new inference from the WebApp. More specifically, the interview is completed and a new inference request is posted. AWS Elastic Beanstalk automates the details of capacity provisioning, load balancing, auto scaling, and application deployment, creating an environment that runs a version of the application.

At a step 24, the recorded data is stored securely in a public cloud storage container, such as an S3 bucket.

At a step 25, the application makes a record of the inference request in its dedicated database.

At a step 26, the application request then triggers an inference request by using a dedicated API. This may be done in an asynchronous manner.

At a step 27, the API gateway validates the request and then calls a Lambda function that actually triggers the inference process.

At a step 28, the Lambda function starts an inference state machine that coordinates the inference process. The inference process is a set of functions that utilize AWS step functions infrastructure for orchestrating the execution, managing dependencies, and the communication between the sub processes.

At a step 29, a state machine keeps track of the status in, for example, a Dynamo database table that can be queried on-demand. The state machine also keeps the status and handles error management of each function.

At a step 30, the state machine extracts the transcript from the audio tracks by using AWS Transcribe. The step function initiates the transcription phase that performs speech-to-text using the AWS Transcribe service.

At a step 31, and using EventBridge, the step function trigger feature extraction requests. Utilizing AWS Eventbridge, the step function triggers the feature extraction sub-processes. EventBridge is a serverless event bus that ingests data from one's own apps, SaaS apps, and AWS services and routes that data to targets.

At a step 32, the event triggers a step function that orchestrates the process of text, audio and video feature extraction. This is a sub-process for the feature extraction across text/audio/video.

Steps 33, 34, 35 describe the different AWS infrastructure components that are used to host the feature extraction functions. Some are done using Batch and some using Fargate depending on the process needs.

At a step 33, a batch job is triggered to extract the features.

At a step 34, the batch job runs on a Fargate compute cluster and leveraging spot instances.

At a step 35, and when completed, the extracted features are uploaded to the S3 bucket.

At a step 36, step functions request inference process in SageMaker using the extracted features.

Steps 37 and 38 are the actual inference. Using the feature extracted in step 110 and the models created as part of the training process, the inference in invoked and the score is calculated and then returned back to the App.

At step 37, the latest published model is used.

At step 38, and on completion, the results are made available to the step function.

Steps 39-42 represent an internal Dynamo DB for the inference process where all processing stats and results are being stored.

At step 39, the step function aggregates the various inference results and stores a combined result.

At step 40, and as the inference process progresses, events are sent to the WebApplication to keep track of the request results.

At step 41, the WebApplication can request the status of an inference process at any time.

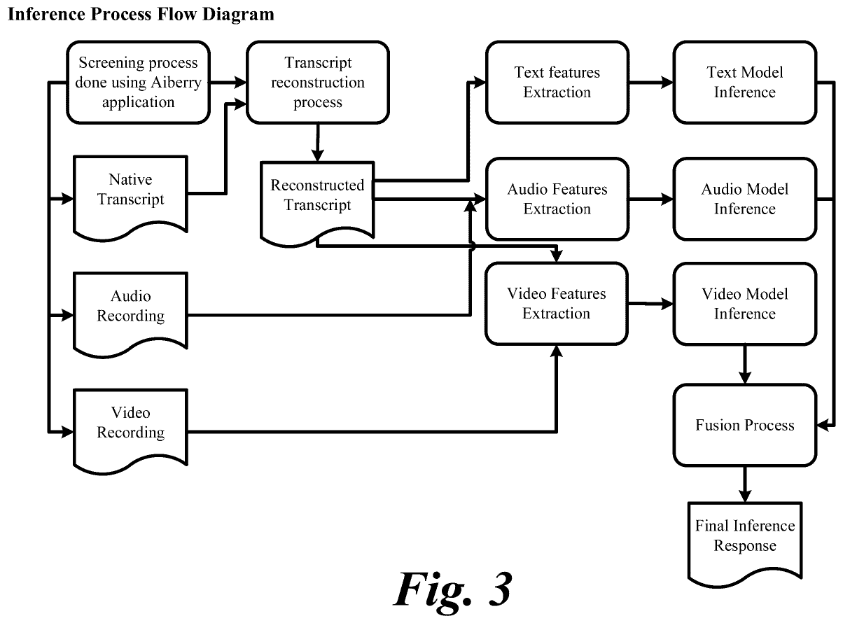

At step 42, the results are retrieved from the inference DynamoDB table. The detailed steps of the inference process are outlined in FIG. 3. The inference process is also designed to work in parallel threads for improved performance and response time.

### Capability 3—Building Database/Dataset for Optimizing AI Training Process.

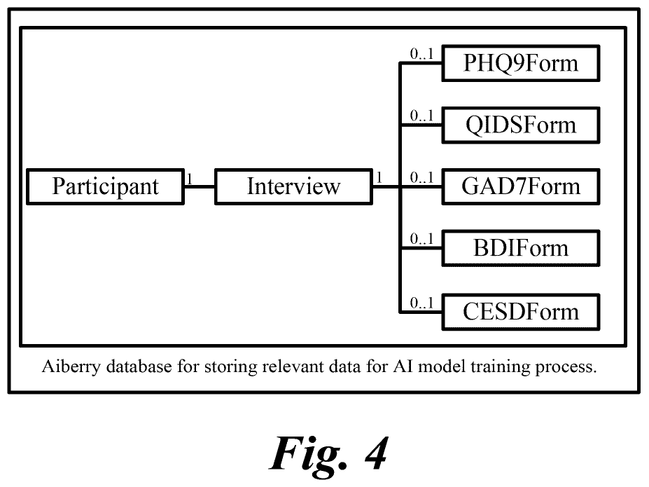

This capability 3 covers the proprietary database according to one or more embodiments developed to store all the data from various input sources. FIG. 4 is an entity relationship diagram that shows the structure of a proprietary database that is used to store data for training purposes. The database has annotated the data and built a data representation that allows for effective AI model training process. The database includes critical information that is used in the training process such as, for example:
Demographic data
Self-reported mental health questionnaires results
Context information captured during the interview process
Locations of all media files
Processing status for each modality
Other specific attributes calculated by the upload process

This information is later used by statistical models for defining and

generating the training K-folds cross-validation which is a statistical method used to estimate the skill of machine learning models (used in the training process). Using the CLI (discussed with reference to FIG. 1) one can extract information from the database based on multiple conditions and search criteria. Based on the information obtained, one can then slice the training population into the training K-folds in a way that mitigates biases e.g. gender, age, symptoms' severity, etc., This capability is advantageous and unique in the way it integrates with the end-to-end training process as it leverages all the data collected to support automatic data extraction and K-folds definition for the AI model training process.

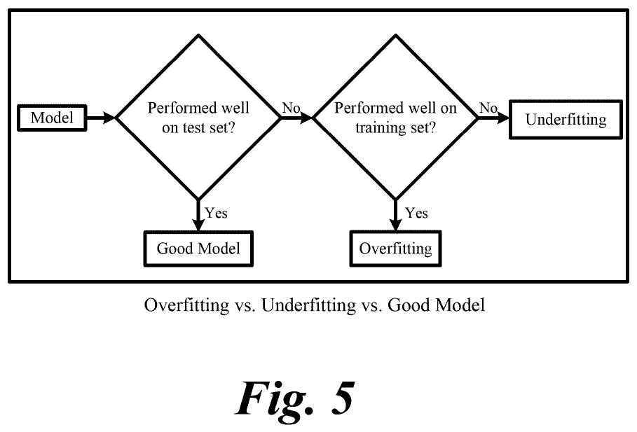

Using this method saves time and is helpful in preparing data for the AI models training that would attempt to mitigate AI models overfitting/underfitting. Overfitting happens when a machine learning model has become too attuned to the data on which it was trained and therefore loses its applicability to any other dataset. Reasons for Overfitting: (a) Data used for training is not cleaned and contains noise (garbage values); (b) The model has a high variance; (c) The size of the training dataset used is not enough; (d) The model is too complex. Underfitting is a scenario where a data model is unable to capture the relationship between the input and output variables accurately, generating a high error rate on both the training set and unseen data. Reasons for Underfitting: (a) Data used for training is not cleaned and contains noise (garbage values); (b) The model has a high bias; (c) The size of the training dataset used is not enough; (d) The model is too simple. FIG. 5 is a simple diagram illustrating the concepts of overfitting and underfitting. The methodology according to one or more embodiments tackles overfitting/underfitting using the following means: (a) Using K-fold cross-validation; (b) Using Regularization techniques; (c) Correctly sizing the training data set; (d) Correctly size the number of features in the dataset; (e) Correctly set model complexity; (f) Reduce noise in the data; (g) Correctly sizing the duration of training the data.

### Capability 4—Multimodal Based Prediction

This capability 4 covers the unique approach of one or more embodiments that leverages a multimodal prediction approach integrating TEXT/AUDIO/VIDEO.

Getting the prediction by using three independent data sources helps improve the accuracy of the prediction of risk levels for certain mental health conditions and detect anomalies and/or problems with less-than-ideal conditions during the screening process.

The solution according to one or more embodiments can include these three modalities: (a) TEXT—Main attribute for the sentiment of what we say; (b) AUDIO—Physical attributes of the way we speak; (c) VIDEO—Facial expressions sentiments that we project while we speak. Each of these modalities has unique ways of extracting features for the training/inference processes. One or more embodiments include a proprietary method for feature extraction to deal with known common problems/challenges in AI models training:

Diarization—Accurate identification and separation of speakers. One objective of diarization is to accurately identifies who says what: what is being said by the interviewer and what is being said by the interviewee. If this process is not done correctly, obviously the chances are that one will encounter further problems in downstream processes.

VAD (Voice Activity Detection)—Cleaning out any non-speaking segments. One objective is to make sure that one can accurately identify and measure periods of speaking vs. periods of non—speaking. This information is advantageous to downstream processes to calculate certain key measurements needed by the inference process.

Preprocessing-Performing dimensionality reduction which is the task of reducing the number of features in a dataset (feature selection). This is advantageous in order to smartly select the right features that will be used in the model training. Too many features or too few features will likely result in the AI model suffering from under/over fitting.

Annotating specific context of the conversation. Annotation of the conversation is an advantageous activity where one can search and mark special markets in the conversation and mark them for downstream processes.

The inference process generates an independent prediction score for each modality and then a proprietary fusion process according to one or more embodiments coupled with the processes described herein combines all those scores into a model which generates the final combined score. This model considers and integrates respective influence/relevancy of each individual prediction score based on statistical data and deduces the final score based on that information. This mechanism is tightly coupled with the AI models and evolves together with the AI models.

Further, a method according to one or more embodiments also assists in tuning the models by understanding the relevant importance/influence of every feature to the scoring prediction accuracy. This is a powerful and beneficial component as it allows the user to further tune the AI models in a methodical and statistically coherent manner.

The information conveyed in FIG. 6 results from one or more embodiments, and is not available from, and cannot be generated by, any prior existing systems. This information is advantageous to the fusion process. As explained before, to one or more embodiments include multiple features that one can extract and use for training across the various modalities. FIG. 6 illustrates the “importance” or weight of each feature compared with other features. That is advantageous for one to tune one's feature selection process and also in the design of one's fusion process. The higher the importance, the higher the significance

The benefits derived from the chart of FIG. 6 are directly associated with the problems of overfitting/underfitting described with respect to Capability 4 discussed herein. The information presented in this chart is generated based on analysis of the AI models performance against the testing data set. This method allows one to judge several aspects around the feature used and as such is very helpful to the model's measurement, training and tuning process.

### Capability 5—Ability to Produce Domain Specific Predictions and Deduct a Final Prediction

This capability covers a unique and proprietary way according to one or more embodiments of generating context-based predictions over each modality.

As part of analyzing data, we have concluded that context is important, and therefore utilize context methodically as an intrinsic part of our analysis, and also integrate detailed observations based on the fact that patients/participants react to different conversation topics differently resulting in various levels of prediction accuracy.

As a result, one or more embodiments include a unique and proprietary method of managing a screening process through a defined set of topics of variable weights. The result is a well-balanced approach between a clinical interview and a casual conversation.

The way this method works is that each question that is being asked during the screening process is mapped into a specific domain and then results are summed up to a specific domain. As a result of this unique approach, one or more embodiments do not only utilize multimodality to get maximum accuracy from independent sources. The solution is also utilizing multiple atomic models across the various modalities and then via fusion process computes the total score. A model according to one or more embodiments consists of three modalities: TEXT/AUDIO/VIDEO. Further, each of those modalities is further segmented into various domains. To be as accurate as possible, each domain within each modality receives a specific score during the inference process. The fusion process is then taking all of those atomic scores and formulating that to a single final inference score.

This method helps to fine tune the overall score accuracy and help to account for high degrees of variability.

FIG. 7 illustrates the process of obtaining from atomic predictions a final combined score via a fusion process which is based on statistical analysis of individual predictions and its specific effectiveness combined with other atomic predictions. This conclusion could not have been discovered or utilized prior to the system according to one or more embodiments. FIG. 7 illustrates the above description. One can see in FIG. 7 how the score is being derived through this navigation tree. In reviewing FIG. 7, one starts at the top of the chart illustrated therein and based on a series of “Yes”/“No” questions one finally ends up in a leaf in the tree that actually illustrates the score. The percentages illustrate the distribution across the population used in this particular process.

### Capability 6—Ability to Track Changes Over Time and Produce Insights/Notifications to Patient and Provider.

This capability covers the capability of one or more embodiments to keep historical records of screening results and allow for the practitioner to analyze changes occurring over a period to give quick context of how screening scores are trending.

The solution according to one or more embodiments also allows for note taking with each screening and those are then presented on a time plot assisting the practitioner to understand the context and potential rationale behind observed changes in scores.

The application also allows the practitioner to filter by screening type and a defined period.

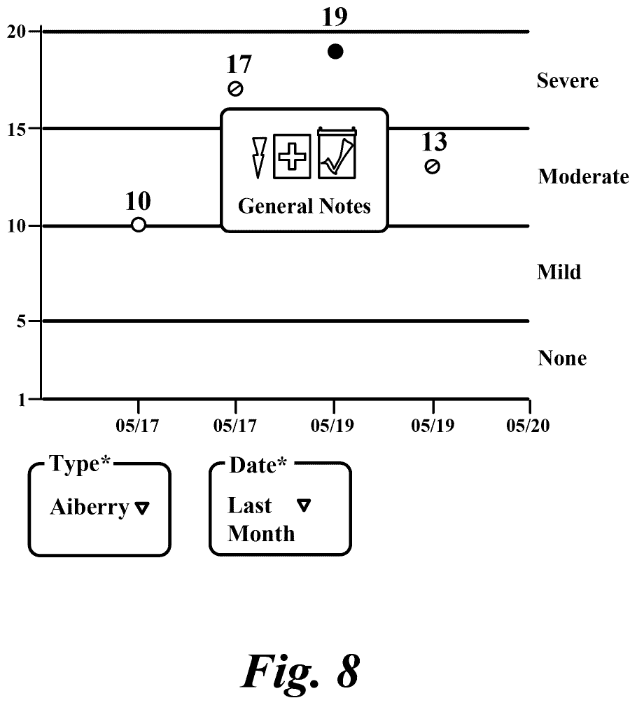

FIG. 8 illustrates the means of plotting patient screening over a period allowing the practitioner to easily view changes in screening scores. In addition to the score, the solution allows practitioners to make notes and annotations for each individual screening which are conveniently visualized on the histogram view allowing the practitioner to quickly build context to potential nature of change across screening. For example, as a result of change in medications or due to a specific stressful event. Putting all of this information at the palm of the practitioner is very helpful and enabling the practitioner in their work.

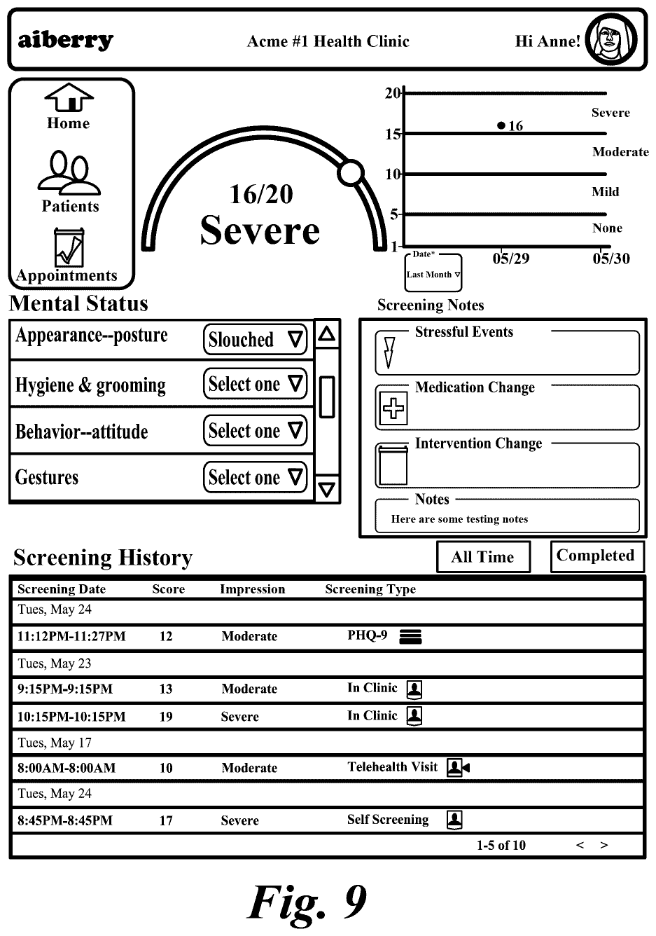

FIG. 9 demonstrates solution capability of keeping track of all historical screenings (left diagram) according to one or more embodiments. Specifically, keeping track of screening score, screening date and screening type. From this view, the practitioner can click on each individual screening entry and get a detailed view (right diagram) which include practitioner notes, and other screening impressions.

### Capability 7—Ability to Identify Inconsistencies Between Self-Reported Scores and AI Based Predictions.

One of the objectives according to one or more embodiments with its AI based screening solution, is to mitigate the problems discussed in the Background section above herein. Using data from studies, we have observed that subjectivity with some participants rating themselves too high or too low versus a clinical analysis of their video interview. The method according to one or more embodiments allows us to identify such cases and highlight those to the practitioner. This has huge value from a clinical point of view as it can help the practitioner to better communicate with their patient and provide them with a less biased score. It can also help the practitioner to establish patterns with patients if they regularly score themselves too high or too low versus the AI based score. To our knowledge, no prior system or analytical tools have been able to objectively quantify in a statistically grounded and legitimate way the subjective screening tools used by clinician practitioners.

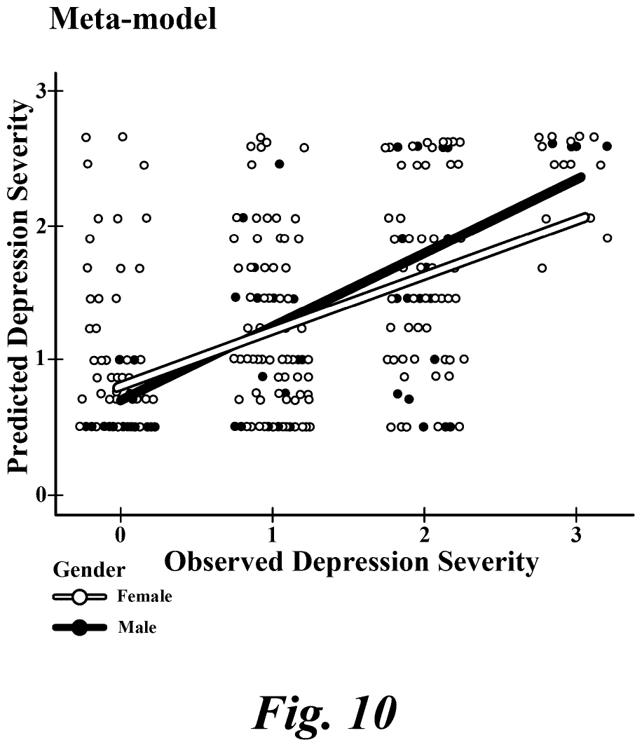

FIG. 10 illustrates the ability of one or more embodiments to identify inconsistencies between self-reported scores and AI-model prediction scores. During data collection one or more embodiments are collecting two pieces of information: 1. A screening interview that is done with any participant in a study. 2. A self-reported standard digital form for the participant to fill out after the screening interview and attest to their situation. According to one or more embodiments one can use PHQ-9 and QIDS-16, which are standard self-reporting digital forms for depression.

Then all the screening interviews are fed to the AI model according to one or more embodiments to get a predicted score and then one compares that score with the self-reported score. Then one can plot all the results on the graph illustrated in FIG. 10 so that one can see the level of discrepancy between the predicted model score vs. self-reported score. This data can then be further analyzed and provided as feedback for an AI modeling team. Ideally, one wants to see a sloped line (as shown in FIG. 10) that shows a great level of alignment.

As illustrated in FIG. 10, the top left quadrant displays inconsistencies between the observed depression scores and the predicted depression scores. For example, when patients self-reported their score to be 0, there is a cluster of predictions between 3 and 1. These inconsistencies require further investigation to classify whether the source of the problem is with the model prediction or the self-reported scores; such investigation and classification are performed by human interaction with the invention. Since one or more embodiments have the capability to produce atomic predictions, this capability is essential for deriving such analysis and correcting inconsistencies.

One or more embodiments provide a method to identify inconsistencies between study participants' self-reported scores and an AI-model scores prediction. One benefit of this approach is during the models training process and another one is during the inference process.

Models training—Being able to flag and analyze inconsistencies in the scores is advantageous to provide some indication to the accuracy of the AI models. Generally speaking, inaccuracies can fall into one or both of two categories. First, it can be an actual problem with the model algorithm or data preparation or features selection process. Alternatively, it can be a case of study participants under- or over-rating their self-reported score, which is not in line with what a clinical review might reveal.

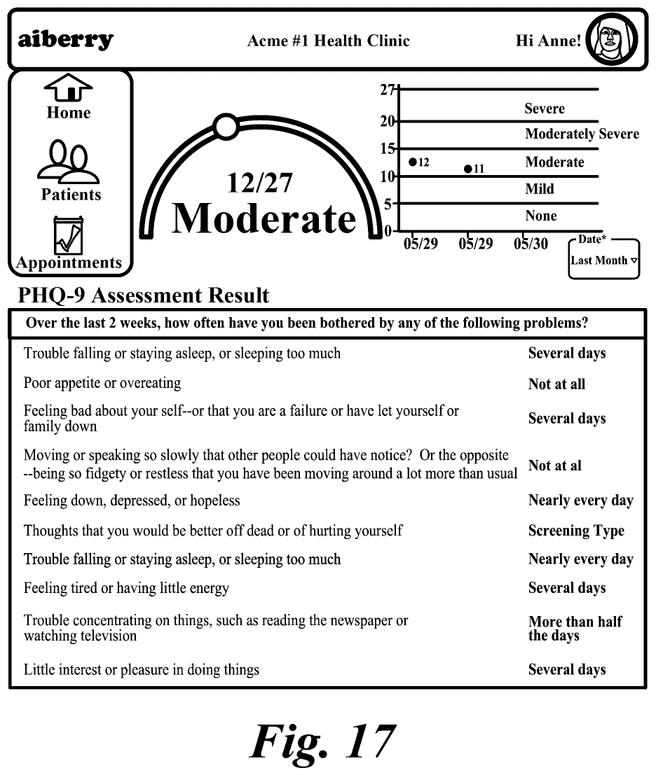

Inference process—In addition to the screening interview, a solution according to one or more embodiments provides a capability for either the patient or the mental health practitioner to ask for a digital form to be filled out in conjunction with the screening. The forms are a digital representation of standard mental health questionnaires such as PHQ-9 as is illustrated in FIG. 17.

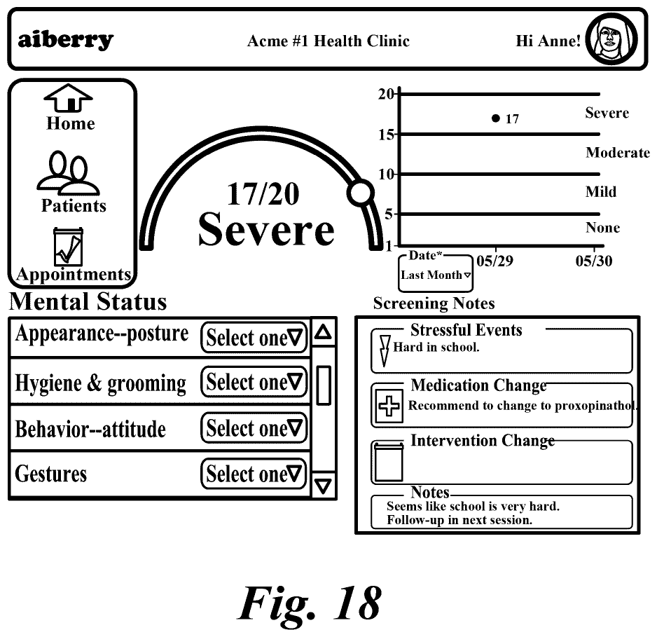

When the AI-based screening is done in conjunction with the digital form request, the solution according to one or more embodiments can then compare the results as illustrated in FIG. 18 and highlight areas of discrepancies.

One or more embodiments include a method to build correlations between digital questionnaire's questions/domains and an AI model according to one or more embodiments and by that identify inconsistencies in responses helping to flag/notify/monitor such occurrences. The digital forms are built around domains, and interviews are built around domains. One then has a mapping between those domains so that one can map back and forth between the two sources.

As such, one can compare not just the total score between the two forms of screening but can actually dive one level lower and understand the source of the differences as it pertains to specific domains. This allows the system to flag such discrepancies and guide the practitioner as to where to investigate further.

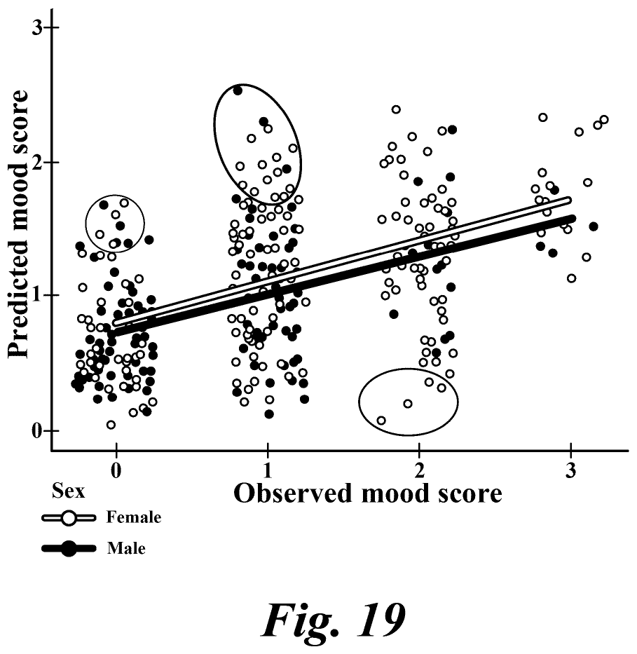

This capability describes a method according to one or more embodiments of identifying and analyzing outliers to help in further tuning the AI models. As outlined in FIG. 19, the infrastructure developed according to one or more embodiments enables the process of identifying, analyzing, and reacting to score outliers. This process is managed as part of the ongoing AI algorithm training process. An objective of this process is to find outliers, then either explain such outliers via clinical review or alternatively determine whether they are the result of a problem in the model that needs to be corrected. Some of the outliers may be legitimate in the sense that via clinical validation one can determine that the predicted score is correct and actually the self-reported score is wrong. Via such validation one can potentially get to higher accuracy than the existing standard tools that are used for training.

The analysis entails both clinical review of the screening interview and detailed comparison of the AI predictions versus self-reported scores in multiple domains and comparing the results across multiple modalities to conclude whether the issue is with the model (and then take appropriate action) or whether it is with the study participant self-reported scores.

One or more embodiments provides the ability to analyze and flag which can help providers better engage with their patients to understand self-view and potentially explore ways of treatment. The purpose of this capability is in terms of how to use the flagged areas of discrepancy not by the AI modeling team but actually by the practitioner. By flagging out the areas of discrepancy between screening according to one or more embodiments and a self-reported form, the system can now flag “suspicious” areas and help the practitioner to direct their attention to further investigate those areas. For example, if someone self-reported very low levels on an energy domain but on the screening, energy came in at a very high level, this might be an area to further investigate to better understand the difference and find out what is causing it from a clinical point of view (e.g., it can demonstrate an issue with how people perceive themselves).

From the practitioner point of view, identifying such correlations and/or discrepancies between the AI based scores and the self-reported scores is valuable as this can be important input for them in how they engage their patients as part of their patient-provider relationship and the care given by the provider.

One or more embodiments include a method developed to identify relevant data for a video sentiment AI model. To better address this area, one or more embodiments include a proprietary method of scanning through study interviews and identifying areas of the videos where there is a major change in participant sentiment. Those sections are then extracted into individual frames, and via frame annotation, a much higher value data set is created that is then trained for sentiment analysis and used by a solution according to one or more embodiments.

A key problem is that there is no formal data based against which one can train a video sentiment analysis algorithm. Most of the frames that are out there are done by actors and they are a clear exaggeration and emphasize certain attributes that in real life scenario and through regular interview conversation do not appear like that. In real life scenarios the cues are much more subtle and as such attempts to train against “stock” pictures is likely to produce bad results.

One or more embodiments involve creating a frames bank extracted from real-life videos and annotating them so then they can be used for training purposes. With that said, even identifying those frames within an existing video is not a simple task and requires a repetitive process to identify->extract->annotate->train->test->analyze->correct->identify.

Once the data bank of pictures is created and verified one can now begin to build an AI model against it. This method is easily expandable should one choose to explore additional sentiments (i.e., features) that one would like to include in the AI process.

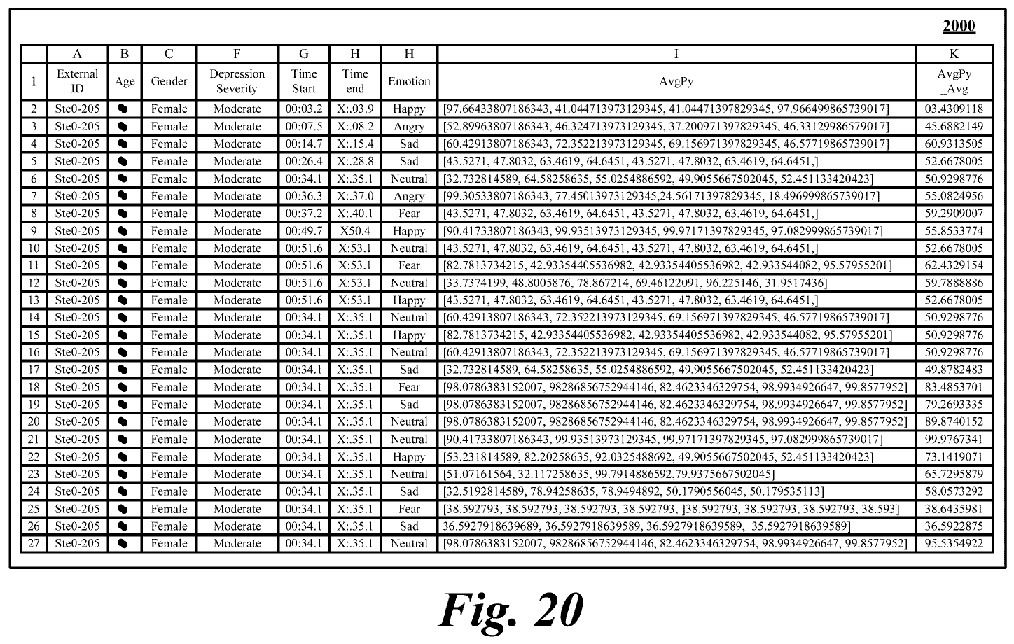

FIG. 20 illustrates a chart 2000 output from a process according to one or more embodiments. The External-ID/Age/Gender/DepressionSeverity fields of chart 2000 are meta data fields that are used in order to then make sure that when the K-Folds are created the data is statistically balanced.

Time_start/_end is the time stamp in the video.

Dmotion is the sentiment focused on.

AvgPr represents the calculated score (0-100 scale) of a specific frame in that segment of the video to actually demonstrate the listed Dmotion. The frames in the segment are sampled every X (set parameter) ms.

AvgPr_avg is the calculated average of the scores presented in the AvgPr column and, as such, gives an overall score for that segment to actually demonstrate the listed Dmotion. Once the data is identified, one can build a data set that will be based on correct statistical distribution of Age, Gender, Depression Severity and Emotions.

One or more embodiments include a mechanism to identify high value visual sections in an interview, correlate to specific domain and extract data to enhance/build a dataset for high quality sentiment analysis AI model based on facial expressions.

Once the data set is created, one can create the gold standard for each emotion based on the data extracted. The next step is to extract all the individual frames and create a picture bank that will then go through a process of frame annotation by a team of experts.

Once all the frames are annotated and verified, those frames can be used to create an AI model for VIDEO sentiment analysis what is in turn used by the solution according to one or more embodiments.

One or more embodiments provide for driving a mental health screening session via an automated chat-bot that may be referred to herein as “Botberry.” As a result of numerous studies, it has become apparent to the inventors of the invention herein that having the correct context in a screening conversation is important so that one can generate the most relevant reactions that are relevant in assessing risk around mental disorders (e.g., depression).

One or more embodiments provide a chat-bot that helps both the mental health practitioner as well as the patient by driving relevant topics in screening conversations for both telehealth and self-screenings. Botberry can react to certain responses of the patient and direct the conversation through its algorithm to ask additional follow-up questions.

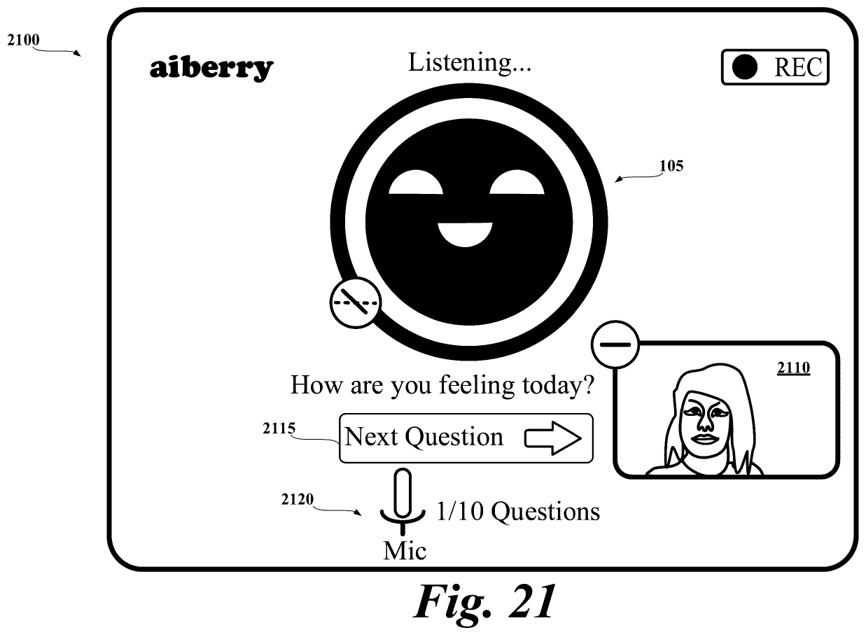

FIG. 21 illustrates an automated chat-bot graphical user interface (GUI) 100 according to one or more embodiments driving a self-screening. Chat-bot Botberry 105 conducts the screening interview and asks questions of the patients. The questions may be a predetermined set of questions or each question subsequent to the first question may be generated in response to and based on the content of the patient's response to each such question. The questions are spoken (i.e., audible to the patient over one or more speakers) as well as displayed on a screen in GUI 100. The patient provides a spoken response to each question via a microphone communicatively coupled to the processing device that provides GUI 100 and executes Botberry 105. Alternatively, the patient may provide a response to each question via a keyboard or similar input device coupled to the processing device.

Window 2110 provides the ability of the patient to see themselves during the interview and ensure that they are being seen and properly located in the frame during the interview so that, for example, video sentiment analysis, either electronic or manual, may be performed on the patient either in real-time or at a later time. Selection with, for example, a pointing device of progression control 2115 enables the patient to move on to a next question once the patient is satisfied that they have sufficiently answered a question currently posed by Botberry 105. A control panel 2120 enables the patient to see how many questions are still pending in the screening interview as well as enabling the patient to set up their input/output communication devices (e.g., microphone, speaker, camera, etc.).

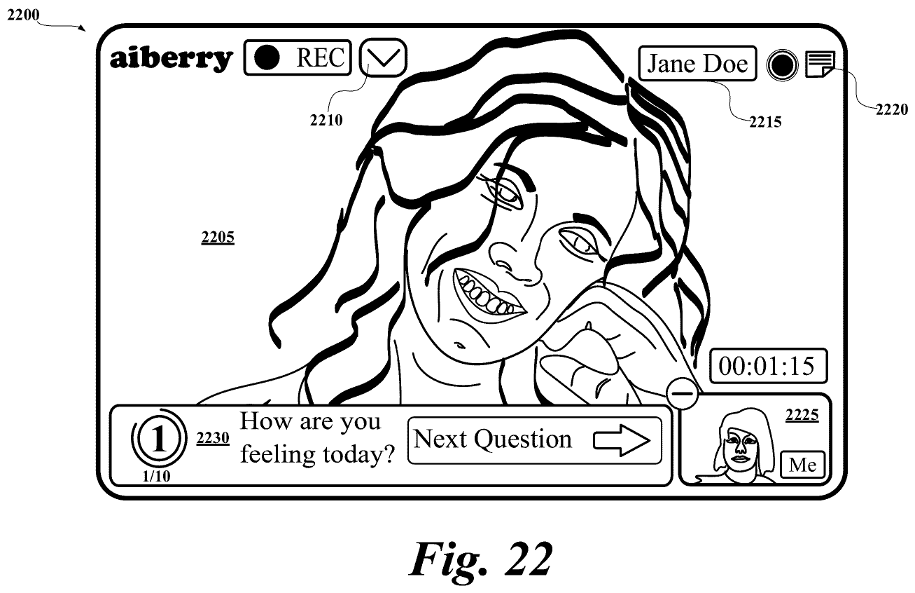

FIG. 22 illustrates an automated chat-bot GUI 2200 according to one or more embodiments driving a telehealth session. Patient screen 2205 provides the ability of the patient to see themselves during an interview with a medical practitioner and ensure that they are being seen by the practitioner and properly located in the frame during the interview so that, for example, video sentiment analysis, either electronic or manual, may be performed on the patient either in real-time or at a later time. A control panel 2210 enables the patient to set up their input/output communication devices (e.g., microphone, speaker, camera, etc.). Identifier icon 2215 provides the name of the patient. Selection with a pointer device by the practitioner of a drawer icon 2220 enables the practitioner to open one or more data-entry fields during the interview process to capture relevant notes pertaining to the interview. Window 2225 enables the practitioner to see themselves and make sure that they are visible to the patient. Prompting panel 2230 enables the practitioner to manage the screening interview. Panel 2230 enables the practitioner to see the questions to ask, how many questions remain in the interview and proceed to the next question once the currently asked question has been sufficiently answered by the patient.

For the patient, this is helpful as it provides an actual interview in a safe environment and drives the conversation. For the practitioner it also helps in driving the conversation by suggesting follow-up questions. Although the practitioner is not bound to the specific questions suggested by the chat-bot and can “go off-script” by adding some follow-up questions of their own, even in such cases the chat-bot operates as a helpful conscious or sub-conscious catalyst to the practitioner.

One or more embodiments include Botberry Smart Scripting with the objective of managing a screening process to drive optimum reactions and more accurate predictions.

This capability covers one of Botberry's functions, which is to drive a screening process to maximize accuracy of mental health disorder predictions. Throughout our studies, we have established that different discussion topics and different questions pertaining to those topics generate different reactions for patients. Some such topics/questions have shown much higher relevancy for predicting mental health disorders while others have less relevancy, and this detailed insight enabled by embodiments herein was not previously available to practitioners.

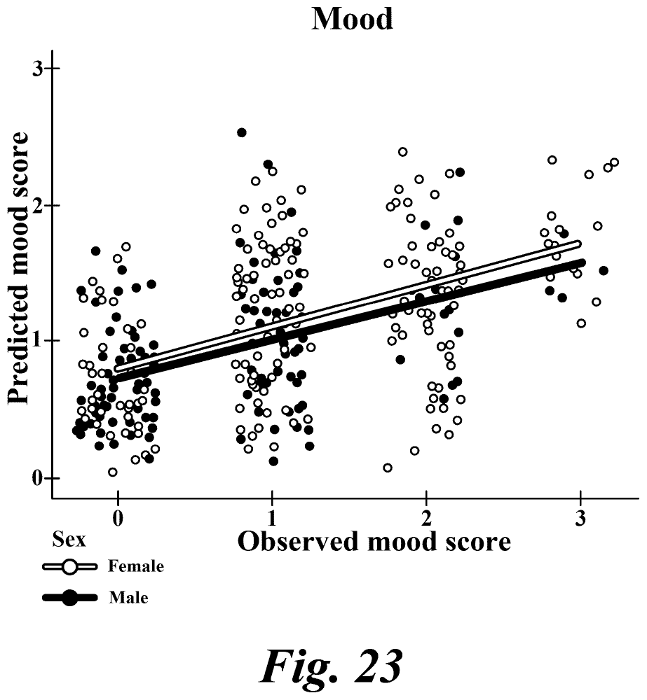

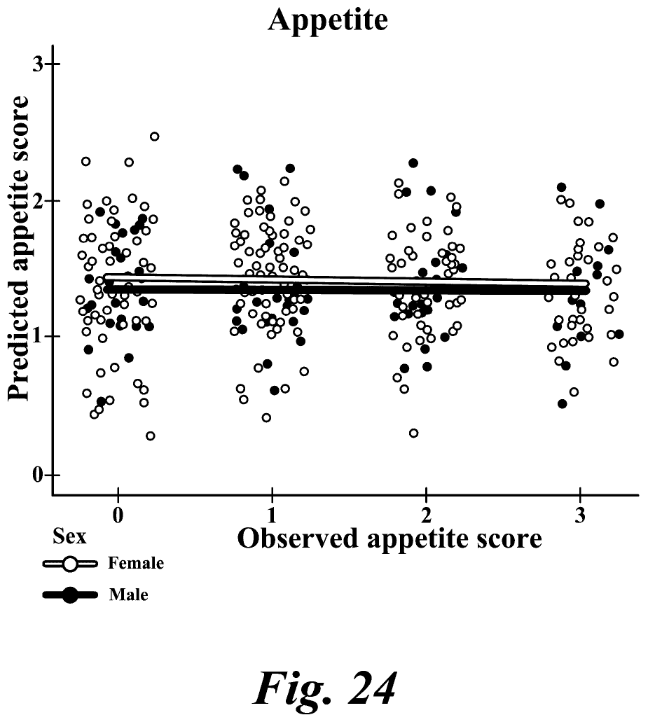

As illustrated in FIGS. 23 and 24, one can see the difference between a high relevancy topic (Mood) showing in FIG. 23 a pronounced slope line (correlation) between scores rating different conditions of the patient as reported by the patient on one axis and AI-predicted scores of such conditions generated based on analysis of one or more interviews using a chat-bot as above discussed. In FIG. 24, a low relevancy (Appetite) topic shows a flat line and, as such, low correlation between patient-reported and AI-predicted scores.

This type of data analysis achieved by one or more embodiments helps drive the conversation topics that are then used by Botberry to drive a screening. The unique approach of use of Botberry is the combination of and right balance between clinical and general conversation approaches to maximize reactions from the patient while keeping the screening process as close to a natural conversation as possible.

To drive Botberry conversations, one or more embodiments include a proprietary algorithm that combines core and optional domains, priority, follow-ups, and several other attributes to build a natural, randomized and clinically sound screening process. For purposes of the discussion herein, the term “domain” may refer in non-limiting fashion to topics of analysis including one or more of Sleep, Appetite, General Wellbeing, Anxiety, Diet, Interests, etc. More specifically, Botberry includes or otherwise has access to a question databank with a variety of questions around different domains such as General-Wellbeing, Sleep, Hobbies, etc. Botberry can pick from this databank of questions the relevant questions for the screening. The selection process is contextual and ensures that the questions will not be too often repeated to the patient, that the topics will change between screening, whether it is a first-time screening or a follow-up screening. Once the selection process is completed, Botberry will administer a screening that is tailored to each patient situation.

A system according to one or more embodiments keeps track in a database which exact questions were used for which patient for every screening. In addition, the system keeps track of the score details and screening insights. All that information, along with the questions bank, is fed into the system to generate the potential questions to be used in the next screening. Every screening, the process repeats itself such that the use of the questions bank can be best optimized and optimally tailored to each individual for the best experience.

One or more embodiments include real-time reaction to patient screening responses to adjust the screening script to “double-click” on certain domains. This capability covers Botberry's ability to respond in real-time to patient inputs and drive the conversation through follow-ups zooming into specific area to collect further inputs. In addition to driving the screening conversation, Botberry can also identify and highlight key words and phrases for the benefit of the practitioner. Botberry uses sentiment analysis and a data dictionary to identify relevant key phrases. In addition, Botberry can use certain attributes to establish the sentiment of the patient responses (e.g., open/closed questions, valence, purpose, etc.). More specifically, during the screening process, Botberry can, in real-time, react to certain responses from the patient and will drill further down into the domain the subject of such responses. As an example, Botberry can administer a suicide ideation risk assessment in response to patient feedback during the depression screening. The databank of questions keeps certain attributes for each question such as the valence, type, purpose etc. which are then later used during the inference process to deduce the sentiment and additional relevant insights. This capability is very helpful in driving a more natural flowing conversation with emphasis on clinical added value of trying to collect additional data points that can be helpful in driving a more accurate prediction.

Based on how the Botberry algorithm is configured, certain types of questions may warrant follow-ups for which Botberry can randomly select a suitable question as a follow-up. Botberry is configurable and can be easily adapted to ongoing developments and insights that one may find through various studies. The screening algorithm can decide based on certain responses whether to focus on specific topics. For example, if during the screening process there are clear signs for potential suicide ideation, the screening algorithm can administer a suicide ideation risk questionnaire to specifically focus on this topic. All resulting information will then become available to the practitioner.

One or more embodiments may include a friendly helper guiding the provider and patient through the platform. This capability covers Botberry's ability to serve as a friendly helper as the provider or patient navigate a platform according to one or more embodiments. Botberry's friendly mannerism guides the users towards functionality that otherwise might be overlooked, especially during the first few times of usage. Botberry can learn user behavior over time and provide less assistance as users familiarize themselves with the platform. This capability functions similar to a tour and allows the user to get familiarized with key features of the platform. As new features are added they can be included in the tour capability and be used as an introduction to the user of new capabilities of a new software release.

This application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms. In addition, the headings in this application are for reference purposes only and shall not in any way affect the meaning or interpretation of the present invention.

Embodiments of the present invention may comprise or utilize a special-purpose or general-purpose computer including computer hardware, such as, for example, one or more processors and system memory, as discussed in greater detail below. Embodiments within the scope of the present invention also include physical and other computer-readable media for carrying or storing computer-executable instructions or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the invention can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems or modules or other electronic devices. When information is transferred or provided over a network or another communications connection (either hardwired, wireless, or a combination of hardwired or wireless) to a computer, the computer properly views the connection as a transmission medium. Transmissions media can include a network or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the invention. The computer executable instructions may be, for example, binaries, intermediate format instructions such as assembly language, or even source code.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

As used herein, a process that is performed “automatically” may mean that the process is performed as a result of machine-executed instructions and does not, other than the establishment of user preferences, require manual effort.

Although the foregoing text sets forth a detailed description of numerous different embodiments, it should be understood that the scope of protection is defined by the words of the claims to follow. The detailed description is to be construed as exemplary only and does not describe every possible embodiment because describing every possible embodiment would be impractical, if not impossible. Numerous alternative embodiments could be implemented, using either current technology or technology developed after the filing date of this patent, which would still fall within the scope of the claims.

Thus, many modifications and variations may be made in the techniques and structures described and illustrated herein without departing from the spirit and scope of the present claims. Accordingly, it should be understood that the methods and apparatus described herein are illustrative only and are not limiting upon the scope of the claims.

What is claimed is:

1. A computer-implemented method, comprising the steps of:
receiving from a user over a network a media file including a recorded patient screening interview;
receiving from the user over the network first data comprising one or more responses provided by the patient to a mental health questionnaire;
generating a transcription of audio associated with the media file;
performing video sentiment analysis on video associated with the media file to generate a second data set; and
based on at least one of the transcription, first data and second data, generating an artificial intelligence model configured to provide predicted risk levels of the patient for one or more mental health conditions.

2. The method of claim 1, further comprising the steps of:
identifying one or more features characterizing one or more responses of the patient included in at least one of the transcription, first data and second data;
associating the one or more features with respective at least one of lengths, colors and shades representing relative variable importance of the one or more features; and
displaying in a graphical display the respective at least one of lengths, colors and shades of the features.

3. A computer-implemented method, comprising the steps of:
accessing a memory device on which is stored a set of questions pertaining to mental health of a patient;
presenting to the patient via a graphical user interface (GUI) a first question of the set of questions;
recording, via a microphone, a first answer of the patient to the first question;
recording, via a camera, images of the patient answering the first question;
based on at least one of the recorded answer and the recorded images, selecting a second question of the set of questions;
presenting to the patient via a graphical user interface (GUI) the second question of the set of questions;
recording, via a microphone, a second answer of the patient to the second question;
recording, via a camera, images of the patient answering the second question; and
based on the first and second answers, determining a score characterizing a relevance of a mental health topic to the patient.

4. A computer-implemented method, comprising the steps of:
receiving from a user over a network a media file including a recorded patient screening interview;
receiving from the user over the network first data comprising one or more responses provided by the patient to a mental health questionnaire;
generating a transcription of audio associated with the media file;
performing video sentiment analysis on video associated with the media file to generate a second data set; and
based on at least one of the transcription, first data and second data, generating an artificial intelligence model configured to provide predicted risk levels of the patient for one or more mental health conditions.

5. The method of claim 4, further comprising the steps of:
identifying one or more features characterizing one or more responses of the patient included in at least one of the transcription, first data and second data;
associating the one or more features with respective at least one of lengths, colors and shades representing relative variable importance of the one or more features; and
displaying in a graphical display the respective at least one of lengths, colors and shades of the features.
